(19) United States
(12) Patent Application Publication
(10) Pub. No.: US 2006/0051544 A1
(43) Pub. Date: Mar. 9, 2006

(54) Implant having a long-term antibiotic effect

(76) Inventor: Helmut Goldmann, Tuttlingen (DE)

Correspondence Address: NATH & ASSOCIATES, 112 South West Street, Alexandria, VA, 22314, US

(21) Appl. No.: 10/521,455

(22) PCT Filed: May 14, 2004
(86) PCT No.: PCT/EP04/05162

§ 371 Date: Jan. 14, 2005

(30) Foreign Application Priority Data
May 15, 2003 (DE) 103236767

(51) Int. Cl. A61F 2/04; B32B 15/08; A61L 27/00

(52) U.S. Cl. 428/35.8; 428/544; 427/2.25; 427/2.24

(57) ABSTRACT

The invention relates to an implant with antibiotic long-term action, in particular a vascular prosthesis, with a basic structure which defines the form of the implant and which is made of substantially non-absorbable or only slowly absorbable polymer material and of a coating of an absorbable material, with a layer of metallic silver situated on the polymer material and underneath the coating.

The invention relates to an implant with antibiotic long-term action. Infection following implantation of prostheses and other implants is a risk factor feared by physicians and patients alike. The incidence of implant infection is approximately 0.5 to 5%. Risk factors affecting artificial vascular implants are, for example, emergency operations, a subcutaneous position of the prosthesis or, possibly, positioning of the prosthesis in the inguinal region. A distinction is drawn between early infections, which generally occur within a period of up to 4 months after implantation, and so-called late infections which become apparent after a longer period of time has elapsed since implantation. Clinical reports confirm, for infections of the aorta for example, an onset after 25-70 months. In the aorto-femoral position, the average time until onset of infection is 41 months. Extracavitary prosthesis infections occur earlier (within 7 months). The microbes which cause such infections include, in particular, Staphylococcus aureus, Staphylococcus epidermidis and Escherichia coli. The infection generally results from intraoperative contamination. However, it can also occur in the post-operative phase, in particular in the case where the patient has an infection that has not completely healed. The microbes or microorganisms tend to adhere to the prosthesis surface. In doing so, they may form a microcolony within a biofilm, and in the course of time they may become sealed off from the outside. Particularly in cases where the patient has been weakened for other reasons, virulent infection and inflammatory reactions may occur with involvement of the perigraft tissue and the anastomosis regions.

It is known that silver has an antibiotic action. Silver salts and metallic silver are therefore widely used to combat microorganisms. Thus, it is known, for example from WO 93/07924, for articles made of plastics, metal and ceramics and introduced into the body, for example fixation devices, nails, pins, catheters, stents, tracheostomy tubes, shunts, percutaneous connectors, wound drainage devices, dental implants and the like, to be provided with a bactericidal component, in particular of platinum, iridium, gold, silver, mercury, copper, iodine and their alloys, compounds and oxides. The corresponding substances are applied in the form of ionized atoms in a vacuum chamber by ion-beam-assisted deposition (IBAD). Biomedical implants with similar bactericidal surfaces are described in U.S. Pat. No. 5,492,763. In the latter, biomedical articles such as metallic needles, urology catheters, percutaneous clamps and ceramic and metallic countersurfaces of hip joints and knee joints are mentioned.

From WO 81/02667, it is also known to provide implants, for example artificial joints, with a surface coating of silver or silver alloys in a layer thickness of 25 to 500 Å, in order, on the one hand, to avoid bacterial growth and, on the other hand, to ensure that the amount of silver is not so great as to damage surrounding connective tissue.

From U.S. Pat. No. 5,464,438 it is also known for metallic gold to be vapor-deposited on implants made of textile material, in order to reduce the risk of thrombosis.

Textile implants, especially when used as replacements for hollow organs, in particular ducts, and chiefly including vascular prostheses, are normally provided with sealing coatings in order to close the pores of the textile prostheses at least initially. It has been proposed to incorporate bactericidal substances into the coating material in order in this way to be able to avoid infections after implantation. Such coatings, which among other things can contain silver ions, are set out in WO 00/32247.

It is an object of the invention to make available an implant, in particular a vascular prosthesis, with antibiotic long-term action, which implant is to be able to be handled in the normal way and will reduce the risk of infection to a minimum.

The subject of the invention is an implant with antibiotic or antimicrobial long-term action, in particular a vascular prosthesis, with a basic structure which defines the form of the implant and which is made of substantially non-absorbable or only slowly absorbable polymer material and of a coating of an absorbable material, with a layer of metallic silver situated on the polymer material and underneath the coating.

It was to be feared that an interaction would take place between the absorbable coating and the silver layer. This is also actually the case especially when the absorbable layer is made of biological material such as gelatin and collagen. However, it was found that this interaction is rather of advantage. Thus, as will be explained in more detail below, comparative tests have shown that the release of silver ions in prostheses provided with an absorbable layer is initially very high compared to prostheses provided only with a silver layer, even when no silver was incorporated in the absorbable layer. The silver layer is evidently corroded by the constituents of the absorbable layer, which can occur during storage of the prosthesis up to the time it is used. Released silver ions deposit in the absorbable layer and, as the latter breaks down, are released more rapidly. If the silver layer is of sufficient dimension, this does not impair the long-term action of the silver layer, with the result that the bactericidal action of the silver layer is maintained for a long time even when the absorbable layer is broken up.

The silver layer advantageously adheres firmly to the surface of the polymer material and is in particular anchored in it. This can be achieved using the vapor-deposition methods known from the prior art, in particular the abovementioned IBAD technique. The silver layer is therefore preferably vapor-deposited onto the polymer surface. It is particularly preferred if the silver atoms of the silver layer are impressed into the polymer surface of the basic structure. This can advantageously be done by bombarding the polymer surface with argon ions, for example, during the vapor-deposition.

The silver layer covers the polymer surface at least at the locations where it comes into contact with connective tissue after implantation, and it preferably covers it completely. Closed silver layers are present in particular at least in these areas. In the preferred embodiment, the silver layer is of such thickness that in vivo, i.e. after implantation, it has a dwell time on the polymer surface of more than one year, in particular of more than 2 years, and releases silver ions during this time. It is particularly advantageous if the silver layer is of such thickness that, as it breaks down in the body, only about 5 to 10%, in particular 7 to 8%, of the layer thickness is removed per annum. It has in fact been found that the possible damage to the surrounding tissue, as described in the literature, is not a function of the layer thickness of the silver layer. Thicker layers do not release more silver ions per unit of time, but as a result they release them for a longer period of time. Layer thicknesses in the range of 1000 Å to 2500 Å have proven useful, in particular those of ca. 1300 Å. Such layer thicknesses exhibit a good long-term action. The layer thickness can also be greater and amount to as much as 4000 Å and over, but greater layer thicknesses do not bring any real additional advantages. Smaller layer thicknesses may, particularly because of the interaction with the absorbable layer, lead to an undesirably early attenuation of the long-term action.

The polymer material for the basic structure can be from the usual polymers used in implants, in particular vascular prostheses, for example polyester, polytetrafluoroethylene, polyurethane and, in special cases, also polyamides, preference generally being given to polyester. The silver layer is preferably situated at least on the side or sides of the polymer material facing toward the connective tissue. The silver layer is preferably composed of pure elemental silver.

The basic structure of the implant is porous, especially in the case of a vascular prosthesis, but also in the case of hernia meshes, patches and the like, and the absorbable layer is an impregnation which seals off the pores of the implant. As has already been mentioned above, the absorbable layer can be formed from biological material which, if appropriate, can be crosslinked. Possible materials are, in particular, collagen, gelatin and albumin. Alternatively, or in combination, the absorbable layer can also be made from synthetic polymers and copolymers which are degradable or absorbable in vivo. In addition to at least partially water-soluble polymers such as polyvinyl alcohol and carboxymethylcellulose, these mainly include the polymers and copolymers of hydroxy acids. In this context, these are in particular polymers and copolymers of glycolide, lactide, ε-caprolactone, trimethylcarbonate and paradioxanone. It is also possible to use mixtures of the polymers. By suitable choice of the polymers, the desired duration of absorption can be set. This is preferably within 4 months and in particular within 40 days. Such a time is expedient since, depending on the type of prosthesis, the impregnating action is no longer necessary during this time because of the ingrowing connective tissue.

The coating of absorbable material, which in the case of a flat implant can be provided on just one side or else on both sides and can also be made of different materials depending on the intended application, can in turn contain active substances which are released into the surroundings during the absorption period. These are mainly active substances other than silver, for example antibiotics with a particular spectrum of action, or growth factors, active substances with hormonal action, and so on.

A porous basic structure is particularly advantageously made from a textile material, as is the case for example in vascular prostheses and hernia meshes. Suitable materials are formed-loop knits, drawn-loop knits, braids, wovens and nonwovens, preference usually being given to formed-loop knits. It is also possible to use combinations of the textile structures, for example formed-loop knits which have a nonwoven cover layer. Porous sintered material, such as expanded polytetrafluororethylene, can also be used as polymer material, and this is a frequently used polymer material especially for vascular prostheses.

The silver layer is preferably a closed silver layer. However, this does not mean that the pores in the case of a porous vascular structure are sealed off by the silver layer. Rather, the silver layer adapts to the surface structure of the polymer material so that the pores retain their original shape and size. This applies for expanded polytetrafluoroethylene in the same way as for textile fiber material. In the case of fiber material, the fiber surface is coated with silver. In the case of textile fiber material, it is possible to provide the fibers or yarns with the silver layer before the basic structure is formed from them. It suffices, however, for silver to be vapor-deposited onto the finished basic structure at the accessible and/or desired locations, since it is these locations which are exposed to the risk of infection and come into contact with the surrounding tissue.

Further features of the invention will be evident from the following description of preferred embodiments in conjunction with the dependent claims. The individual features of one embodiment can in each case be realized singly or severally.

### EXAMPLE 1

Double-velour knitted prostheses of polyester are clamped in a rotatable clamp device so that they hang freely as a bundle of parallel tubes with spaces between them. The clamp device is introduced into a vacuum chamber suitable for carrying out the IBAD technique, the vascular prostheses being vapor-deposited with silver and at the same time bombarded with argon ions. The coating operation is conducted until a silver layer thickness of 1300 Å is reached on the outside of the vascular prostheses or the fibers located there. If so desired, a primary coating can be effected by vapor-deposition of other metals. Silver is also forced into the pores or interstices between the fibers of the vascular prostheses, so that the fiber surfaces are coated at these locations too. However, the layer thickness is less there because of the “shadow effect” in the vapor-deposition.

The vascular prostheses coated in this way are removed from the clamp device and then impregnated in the usual manner with absorbable material at least on their outside, sealing off the porous structure. This impregnation can be done in the usual way with collagen, in which partial crosslinking with glutaraldehyde is effected. Preference is given to a likewise known coating with gelatin which is crosslinked with diisocyanate. As has been mentioned, bioactive substances can be introduced into the coating solution in order to develop the biological activity during the later absorption of the layer.

Determination of the amount of silver on the vascular prostheses (still without absorbable layer) has revealed that the proportion of silver relative to the total weight of the metallized prosthesis lies in the range of from 0.4 to 0.8% by weight. The proportion of silver depends inter alia on the porosity of the basic structure of the vascular prosthesis. Close-knitted structures have a lower percentage proportion of silver than more porous structures. Moreover, the penetration of the porous implant with silver can be influenced by the way in which the method is carried out, for example by moving the implants during vapor-deposition, by guiding the streams of vapor and gas in a particular way, etc. If, for example, an inner coating of tubular prostheses with silver is also desired, silver vapor can also flow through the inside of the prostheses during the coating operation. Turning the prosthesis round prior to a repeated vapor-deposition also leads to an inner coating.

Comparison Test

A vascular prosthesis according to Example 1, but not yet provided with the absorbable impregnation layer, was placed in phosphate buffer (pH 7.4) at 37° C.; the phosphate buffer was changed daily and the silver content in the previous phosphate buffer sample was determined. The test extended across a period of 365 days. The silver content in the removed phosphate buffer was initially 35 microgram/l and then fell rapidly, and then after 50 days slowly (15 microgram/l), and after 365 days it was ca. 5 microgram/l.

Under the same conditions, a vascular prosthesis according to Example 1 was examined which was coated with an absorbable impregnation layer of gelatin crosslinked with diisocyanate. Although no silver was added to the gelatin, a high content of silver in the range of ca. 70 to 80 microgram/l was initially found in the phosphate buffer, and although it decreased slightly it remained high until the absorbable layer had largely broken up. It was not until after about 50 days that the silver content in the phosphate buffer had fallen to the level shown after 50 days by the vascular prosthesis not provided with the impregnation coating, after which time the release of the silver ions into the phosphate buffer was essentially the same as in the vascular prosthesis without impregnation coating.

This comparison shows that the silver layer was attacked via the impregnation coating, and silver ions were released into the impregnation coating, and these then entered the phosphate buffer at an increased rate and in increased number. The vascular prosthesis provided with the impregnation layer thereafter showed a comparable release of silver ions, which means that the initial strong release of silver has no negative effect on the long-term action.

### Tissue Reaction

Vascular prostheses produced in a similar way, but with silver layers of 1600 Å and 2500 Å, were implanted in rats, rabbits and pigs. Upon explantation after 3 months and 6 months, good integration was found. All the implants showed no abnormal findings. The internal organs too showed no abnormal findings. There were no signs of chronic inflammatory reactions.

### Artificial Infection

A comparison was conducted using implants according to the invention, and implants which, instead of having a silver layer on the basic structure, contained silver acetate incorporated in the absorbable coating. The comparison specimens were artificially infected with problem microbes and implanted in rabbits. They were explanted after 7 days. The comparison specimens were then incubated for 48 hours in CASO broth, after which a microbial count was conducted. The microbial colonization was determined microbiologically in 36 specimens. It was found that, in the implants according to the invention, only 22%, i.e. 8 implants, were colonized with a small number of microbes, whereas, in the implants with silver acetate in the absorbable coating, infection was found in 67%, corresponding to 23 implants.

1-17. (canceled)

18. Prosthesis for replacement of hollow organs with antibiotic long-term action with a basic structure which defines the form of the prosthesis and which is made of substantially non-absorbable or only slowly absorbable polymer material and of a coating of an absorbable material, with a layer of metallic silver situated on the polymer material and underneath the coating.

19. The prosthesis as claimed in claim 18, wherein the silver layer adheres firmly on the polymer material.

20. The prosthesis as claimed in claim 18, wherein the silver layer is vapor-deposited onto the polymer surface.

21. The prosthesis as claimed in claim 18, wherein silver atoms of the silver layer are impressed into the polymer surface of the basic structure.

22. The prosthesis as claimed in claim 18, wherein the silver layer is a substantially closed layer.

23. The prosthesis as claimed in claim 18, wherein the silver layer is of such thickness that, as it breaks down in the body, a maximum of about 5 to 10% of the layer is removed per annum.

24. The prosthesis as claimed in claim 18, wherein the silver layer has a layer thickness of 2500 to 1000 Å.

25. The prosthesis as claimed in claim 18, wherein the silver layer is composed exclusively of elemental silver.

26. The prosthesis as claimed in claim 18, wherein the basic structure is porous, the silver layer leaves the pores open, and the absorbable layer is an impregnation which seals the pores of the prosthesis.

27. The prosthesis as claimed in claim 18, wherein the absorbable coating is formed from optionally crosslinked biological material.

28. The prosthesis as claimed in claim 18, wherein the absorbable coating is made of synthetic polymers and copolymers which are absorbable in vivo.

29. The prosthesis as claimed in claim 18, wherein the composition of the absorbable coating is chosen such that it is absorbed at the latest after four months.

30. The prosthesis as claimed in claim 18, wherein the coating of absorbable material in turn contains active substances which are released during absorption of the absorbable coating.

31. The prosthesis as claimed in claim 18, wherein the basic structure is made from a textile material.

32. The prosthesis as claimed in claim 31, wherein the fibers of the textile basic structure are coated with silver at least at the locations which point toward at least one surface of the prosthesis.

33. The prosthesis as claimed in claim 32, wherein substantially the entire surface of the fibers being coated with silver.

34. The prosthesis as claimed in claim 18, wherein the basic structure is made from a sintered material.

35. The prosthesis as claimed in claim 18, wherein said implant is designed as a vascular prosthesis.
